(19) United States
(12) Patent Application Publication
(10) Pub. No.: US 2025/0066835 A1
(43) Pub. Date: Feb. 27, 2025

(54) Microbial Image Analysis Method

(71) Applicants: Hitachi High-Tech Corporation, Minato-ku, Tokyo (JP); IHU Mediterranee Infection, Marseille Cédex 05 (FR)

(72) Inventors: Akiko HISADA, Tokyo (JP); Erino MATSUMOTO, Tokyo (JP); Yuusuke OOMINAMI, Tokyo (JP); Didier RAOULT, Marseille Cédex 05 (FR); Jacques BOU KHALIL, Marseille Cédex 05 (FR)

(21) Appl. No.: 18/723,523

(22) PCT Filed: Dec. 29, 2021
(86) PCT No.: PCT/FR2021/052468

§ 371 Date: Jun. 24, 2024

(51) Int. Cl. C12Q 1/18; G06T 7/00; G06V 20/69

(52) U.S. Cl. CPC C12Q 1/18; G06T 7/0012; G06V 20/693; G06V 20/695; G06V 20/698; G06T 2207/10056; G06T 2207/30024; G06T 2207/30242

(57) ABSTRACT

The purpose of this invention is to provide a microbial image analysis method which enables us to obtain information about a microscopic structure of an individual microbe by electron microscope's resolution, and to evaluate a ratio of microbes.

The method of this invention's one aspect comprises a first step for obtaining an image of a specimen in which a sample including microbes is stained, by using an electron microscope; a second step for obtaining a brightness profile regarding a brightness distribution range of the image; a third step for setting a first standard brightness range which meets a first condition regarding a brightness out of the profile up as a region where a first microbe group exists, and setting a second standard brightness range which meets a second condition regarding a brightness out of the profile up as a region where a second microbe group exists; and a fourth step for identifying individual microbes which exist in each of the first and the second standard brightness ranges and/or for calculating a ratio of the microbe which exists in each of the first and the second standard brightness ranges.

### BACKGROUND OF THE INVENTION

### 1. Field of the Invention

The present invention relates to a microbial image analysis method.

### 2. Description of Related Art

With concerns about the worldwide spread of antimicrobial resistance bacteria, identification of an infectious disease-causing microbe and an infectious disease test such as an antimicrobial sensitivity test and the like become important. Here, as a method for identifying a microbe and measuring sensitivity to an antimicrobial, known is a drug sensitivity test method in which a microbe in a liquid is incubated with a stain and then collected by filtration on a filter, and a viability test is used for counting a microbe stained with a substance that stains both a living cell and a dead cell in a field of view of an optical microscope and a microbe stained with a substance that stains only a dead cell (refer to JP-A-2001-509008).

### SUMMARY OF THE INVENTION

According to JP-A-2001-509008, there is a problem that only a method using an optical microscope is disclosed and finer morphology of a microbe is not observed.

Therefore, an object of the present invention is to provide a microbial image analysis method that can evaluate a ratio of a microbe by obtaining information on fine morphology of an individual microbe with resolution of an electron microscope.

A microbial image analysis method according to one embodiment of the present invention includes: a first step for obtaining an image of a specimen in which a sample including microbes is stained, by using an electron microscope; a second step for obtaining a brightness profile regarding a brightness of the image; a third step for setting a first standard brightness range which meets a first condition regarding a brightness out of the profile up as a region where a first microbe group exists, and setting a second standard brightness range which meets a second condition regarding a brightness out of the profile up as a region where a second microbe group exists; and a fourth step for calculating a ratio of the microbe which exists in each of the first and the second standard brightness ranges.

According to the present invention, it is possible to provide a microbial image analysis method that can evaluate a ratio of a microbe by obtaining information on fine morphology of an individual microbe with resolution of an electron microscope.

### BRIEF DESCRIPTION OF THE DRAWINGS

FIG. 1 is a diagram illustrating a microbial image having a different image brightness and an image brightness range;

FIG. 2 is a diagram illustrating an example of the image brightness range;

FIG. 3 is a diagram illustrating an example of microbial image classification;

FIG. 4 is a diagram illustrating a viability test result of a microbe after storage treatment;

FIG. 5 is a diagram illustrating a viability test result of a microbe exposed to oxygen;

FIG. 6 is a diagram illustrating an electronic stain; and

FIG. 7 is a diagram illustrating an image analysis result of a microbe treated with an antibacterial agent.

### DESCRIPTION OF EMBODIMENTS

A background of the present invention will be described prior to description of an embodiment.

As a first viewpoint of a background technique, one of the important basic techniques is microscopic observation, which uses an image of an individual microbe and a group of microbes. For example, in a clinical examination, a difference in a cell wall structure of bacteria is stained with a stain and observed with an optical microscope, such that gram-positive and gram-negative bacteria are separated and classified into a bacillus and a coccus based on an appearance of the bacteria, and information related to bacterial species such as staphylococcus, streptococcus, and the like can be obtained from an approximate shape of aggregates of isolated and cultured bacteria.

In addition to the identification by such staining, it is expected to acquire information on fine morphology of the individual microbe. However, resolution of an optical microscope generally used in the clinical examination is about several hundreds of nanometers, such that even though it is possible to obtain the approximate shape of the microbe and stain information thereof, it is not easy to identify a difference in morphology between the individual microbes.

Here, an electron microscope is used. Since a wavelength of an electron beam is much shorter than that of visible light, a high-resolution image can be obtained, and even fine morphology that cannot be captured with the optical microscope can be observed. Examples of the fine morphology include a structure peculiar to species of the microbe, a structure viewed during cell division, abnormal morphology caused by an influence of a drug and the like.

Since an electron microscope of related art requires a large-scale mechanism such as a mechanism that applies a high voltage to generate a stable electron beam, a mechanism to keep the inside of the microscope in a vacuum state to prevent scattering of the emitted electron beam, and the like, it is difficult to use the electron microscope of related art in a daily microbial test such as a clinical practice or the like. However, in recent years, a desktop electron microscope, which is obtained by improving the electron microscope of related art, is developed and is also expected to be applied to a microbial sample.

One of the problems with an observation method using the electron microscope is that a stain method is limited. For the optical microscope, stains corresponding to various purposes are developed as cell biology, histology, pathology, and the like are improved, and various types of stain methods for staining a component and a function of a cell and a tissue with different colors are established.

For example, the electron microscope uses a method for visualizing a structure of the entire microbe such as a cell membrane, a nucleus, and the like as contrast of black and white images by treating a sample with a stain containing a heavy metal such as uranium, lead, platinum, osmium, and the like, or a method for visualizing localization of a specific molecule with a gold fine particle label. However, there are few reports of stains staining the specific component of the microbe and the function thereof and having contrast with an electron beam.

As described above, while it is desirable to use a method that combines both staining with the optical microscope and high-resolution observation with the electron microscope, there is no practical method in the daily microbial test.

An example of a second viewpoint of the background technique is a viability test of the microbe, which is one of the important test items in the microbial test. For example, when developing a treatment method for a drug, heat, gas, and the like to remove the microbe, or when selecting a therapeutic agent for infectious diseases and the like, viability of a microbe in a sample treated with these treatments is measured, thereby determining a type of treatment, a treatment method, a treatment intensity, treatment concentration, and the like.

There are various viability test methods, and a standard method is a culture method such as a colony forming unit and the like. In the evaluation by colony formation, a certain amount of a microbial suspension of known concentration is smeared thinly on an agar medium and cultured for about a day, the number of colonies of bacteria growing within a certain period of time is regarded as the number of bacteria surviving at the start of culture, a colony forming unit (CFU/ml) is calculated from the number of colonies with respect to a liquid amount of seeded bacteria, and the calculated colony forming unit is considered as the viability at the start of culture.

The culture method is the most widely used method for testing the viability of the microbe. However, there are problems that it takes one or more days for performing the culture to form the colony, a trained judge is required to visually determine the colony formation, and the culture method cannot be applied to a microbe of which culture conditions such as a medium type and the like are unknown.

In order to solve the above-described problems of the culture method, a method using the optical microscope and the like not requiring the culture is developed. Examples of the method include a method for detecting a function such as enzyme activity of a living cell and the like with a chromogenic substrate, a method for performing staining and detection with a difference in substance permeability of cell membranes of a living cell and a dead cell, a method for detecting a morphological change, and the like. The above-described methods are excellent in speed because it is not required to wait for the culture time until the bacteria grow and form a visible colony.

Here, in combination of the above-described first and second viewpoints, pieces of information on the fine morphology of the microbe with the resolution of the electron microscope, such as a structure peculiar to species of the microbe, a structure viewed during cell division, abnormal morphology caused by an influence of a drug and the like, are grasped, and usefulness of a technique for performing the viability test on the same specimen is also considered. For example, a type and a state are grasped from morphological information of a microbe forming a biofilm, treatment is performed on the biofilm with an antimicrobial, a bactericide, and the like that affect the viability of the microbe, and viability of a treated specimen and viability of an untreated specimen are compared with each other, thereby making it possible to evaluate an effect of the antimicrobial and the bactericide in situ (in-situ observation). However, the viability test method using the electron microscope is not put into practical use in microbial tests such as clinical and environmental tests.

The reason why the viability test using the electron microscope is difficult is that in the case of a general electron microscope, there is an observation pretreatment for preserving the morphology of a microbe to be observed in vacuum. Since a protein cross-linking agent such as glutaraldehyde and the like is used in advance to chemically fix the microbe and then dry the microbe, at the time of fixing the microbe, the microbe loses a viability function. Therefore, it is not possible to perform a viability test as described in the optical detection example that visualizes a viability function of an actually living microbe such as a difference and the like in enzyme activity and biological membrane permeability by staining.

Therefore, as one of the methods for observing fine morphology and determining life and death with the same specimen, there is a method for determining morphology related to the life and death of the microbe, for example, a method for determining membrane damage, collapse of an intracellular microstructure, and the like with observation of an ultra-thin slice by using a transmission electron microscope. However, this method requires a skilled technique and laboriousness. In order to calculate the viability by observing a statistically significant number of microbes, an enormous amount of work is required, such that it is difficult to apply this method to the daily microbial test.

An example of another method includes correlative light and electron microscopy in which a stained image obtained by the optical microscope and a morphological image obtained by the electron microscope are associated with each other. In this method, while it is possible in principle to process the same sample and observe the same sample with the electron microscope after determining whether the sample is alive or dead with the optical microscope, operations such as sample preparation, image superimposition, and the like are not simple, and thus difficult to be applied to the daily microbial test.

In the following embodiments, as a technique for grasping the information on the fine morphology of the microbe with the resolution of the electron microscope and performing the viability test with the same sample, a phosphotungstic acid (hereinafter, referred to as PTA) aqueous solution is used as a stain that strongly adheres to a microbe receiving a morphological damage related to life and death of the microbe, and an image containing composition contrast is acquired with backscattered electrons of a scanning electron microscope (hereinafter, referred to as a SEM), thereby providing a method for evaluating a ratio of the microbe corresponding to the viability.

Here, the ratio is acquired in such a manner that when a microbe in a suspension and the like is stained with a PTA aqueous solution, a difference in staining intensities between the microbe not receiving the morphological damage related to the life and death of the microbe and the microbe receiving the morphological damage related to the life and death of the microbe is detected as a difference in image brightness, and each microbe is quantified. This ratio is defined as the viability according to the present invention. As described above, since the observation pretreatment such as fixing and the like is performed, the ratio of living microbes at the time of SEM image acquisition is not indicated, but the embodiment shows that the ratio equivalent to the viability by a method of related art is obtained.

A process of using PTA as a stain in the present invention will be described. In general, in electron staining at the time of observing a biological sample with the electron microscope, stains such as an aqueous solution of uranium acetate, an aqueous solution of lead salt, platinum blue, and the like are often used. These stains are excellent stains that can visualize intracellular structures such as a cell membrane, a nucleus, and the like with contrast of an image by using a difference in affinity with a component forming the cell, but are not used for a stain method for identifying the life and death of the microbe. Uranium is a radioactive substance and is restricted to its availability and handling. Uranium, lead, and platinum are not stains suitable for the daily microbial test due to its toxicity, solution stability, reagent price, and the like.

Therefore, with respect to the selection of the stain, since PTA is a reagent that is relatively inexpensive, easy to be stored, and easy to be used as described below, the inventor considers that PTA could be applied to electronic staining of the microbe in the daily microbial test. PTA is widely used for microscopic sample preparation. For example, PTA is used as a mordant of staining for an optical microscope sample in a pathological examination, and used as a negative stain for observing the microbe with the electron microscope. It is known that PTA is also used for positive staining at the time of observing the cell with the electron microscope, and PTA can separately stain basic proteins, glycoproteins, and polysaccharides by adjusting concentration and pH of a solution and bind them to a carbohydrate portion of glycoprotein. It is reported that when PTA is used for the SEM observation of bacteria, gram-positive bacteria and gram-negative bacteria have different staining intensities.

Here, the inventor finds out that after bacteria with known sensitivity is treated with an antibacterial agent showing a bactericidal action that damages a cell membrane and a cell wall, a specimen stained with PTA is prepared, a backscattered electron image of the SEM is obtained, individual bacteria having high image brightness are identified in the specimen, and a ratio of individuals having high image brightness increases as antibacterial treatment time elapses.

It is known that since the backscattered electron image has a characteristic that contrast by composition of a substance can be obtained in addition to surface shape contrast of a specimen, more PTA is bound to bacteria with a cell membrane and a cell wall damaged by the bactericidal action of the antibacterial agent than live bacteria. This result suggests that the morphological damage related to the life and death of the bacteria can be identified with PTA staining.

Therefore, the inventor carefully reviews a sample preparation method including a PTA staining step and a condition for observing backscattered electrons using the SEM. As an example, a specimen is prepared by placing a microbe treated with a bactericide and the like and a control microbe not treated with the bactericide on equipment. The specimen is fixed with glutaraldehyde of 2.5% for 5 minutes, the specimen is stained with a PTA aqueous solution of 10% by weight concentration for about 2 minutes, and a backscattered electron image is acquired at an acceleration voltage of 5 kV to 10 kV by using the SEM.

As a result, the microbe contained in the specimen treated with the bactericide is mainly classified into two groups, a microbe having high image brightness and a microbe having low image brightness. The image brightness of the latter microbe having the low image brightness is the same as that of the image brightness of the control microbe specimen not treated with the bactericide. That is, it is found out that in a microbial group treated with the bactericide, it is possible to identify, by staining, bacteria receiving the morphological damage caused by the bactericidal action and bacteria not receiving the morphological damage, and to determine a ratio.

Based on the above-described findings, the inventor provides a method for grasping a type of a microbe and a morphological characteristic thereof with an electron microscope, and for easily identifying a microbe receiving a morphological damage related to life and death and a microbe not receiving the morphological damage by image brightness derived from an intensity of electron staining using PTA.

Hereinafter, detailed embodiments will be described. A procedure, drug concentration, a treatment time, an image analysis procedure, and the like described below are examples, and do not limit the scope of the present invention.

### FIRST EMBODIMENT

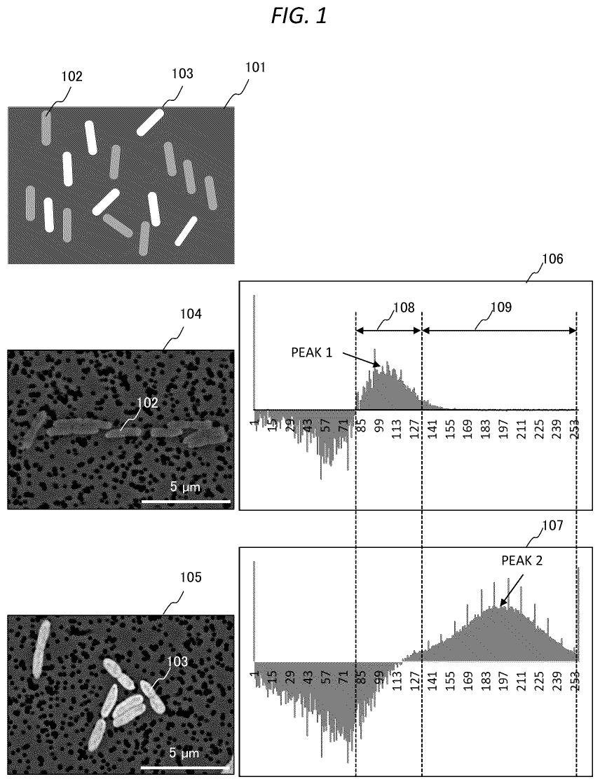

A first embodiment shows an example of classifying a microbial image based on a difference in image brightness of a stained microbe in an electron microscope image of the microbe. FIG. 1 illustrates an example in which a microbe 1 (102) having weak staining and low image brightness and a microbe 2 (103) having strong staining and high image brightness are mixed by using an electron microscope image schematic diagram (101) of a specimen in which the microbe is placed on equipment and stained. In this example, the microbial image is classified into two groups including the microbe 1 and the microbe 2 based on the difference in image brightness.

An example of an electron microscope image (104) in which the microbe 1 (102) exists and an example of an electron microscope image (105) in which the microbe 2 (103) exists are backscattered electron images obtained in such a manner that a Pseudomonas aeruginosa strain is placed on a polycarbonate track-etched membrane, the Pseudomonas aeruginosa strain is fixed with glutaraldehyde of 2.5% and stained with a PTA aqueous solution of 10% by weight concentration, a specimen in which Pseudomonas aeruginosa is placed on the membrane is inserted into an SEM sample chamber, an SEM acceleration voltage is set to 5 kV, and brightness contrast setting at the time of image generation is set to be constant. Since SEM image generation conditions are constant, the difference in brightness between the microbe 1 (102) in the image (104) and the microbe 2 (103) in the image (105) is derived from a difference in a PTA staining intensity.

As an example, the track-etched membrane made of a plastic material is used as the equipment, and the equipment is not limited thereto. Here, from a viewpoint of image analysis later, it is desirable that a user selects equipment made of a material having image brightness different from that of the stained microbes 1 and 2 to be analyzed in the backscattered electron image.

A brightness profile (106) related to the brightness of the image (104) and a brightness profile (107) related to the brightness of the image (105) are difference histograms obtained in such a manner that the number of pixels for each brightness is obtained for each image in which the microbe 1 or the microbe 2 is placed on the membrane, and the number of pixels for each brightness in a membrane image in which the microbe is not placed is subtracted therefrom.

For example, an image region of the microbe 1 is characterized with a standard brightness range 1 (108) including a peak 1 of the brightness profile (106) with respect to the brightness, an image region of the microbe 2 is characterized with a standard brightness range 2 (109) including a peak 2 of the brightness profile (107) with respect to the brightness, such that the images of the microbe 1 and the microbe 2 are classified.

As described above, when the brightness contrast of the SEM is adjusted to be constant at the time of acquiring the image (104), the image (105), an image in a different region of the same specimen, and images of a plurality of specimens made with the same material and method, the standard brightness range 1 (108) that characterizes the image region of the microbe 1 and the standard brightness range 2 (109) that characterizes the image region of the microbe 2 can be set with the same value, when image analysis is performed.

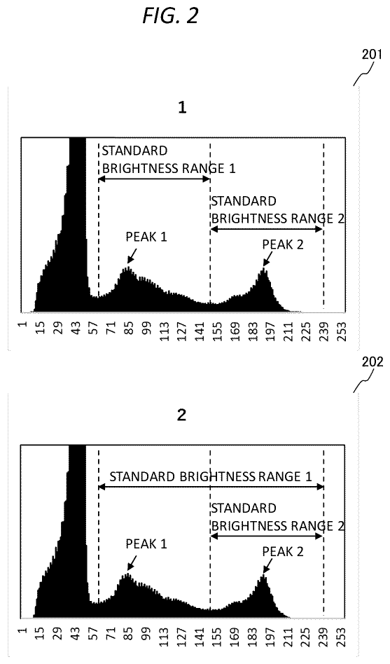

FIG. 2 illustrates examples (201 and 202) of setting the standard brightness range. 201 and 202 are the same histograms and are brightness profiles related to the brightness of the electron microscope image. As illustrated in the schematic diagram (101) of FIG. 1, since 201 and 202 are derived from the image in which microbes having different image brightness are mixed, the peak 1 and the peak 2 exist in the examples of 201 and 202.

Another peak located to the left of the peak 1 corresponds to an electron microscopic image region of the equipment in which the microbe is placed, and thus this range is excluded to determine the standard brightness range that characterizes the microbial image.

As shown in the histogram 1 (201), the standard brightness range 1 including the peak 1 derived from an image of the microbe 1 is determined, and a standard brightness range including the peak 2 derived from an image of the microbe 2 is determined within a range that does not overlap with the standard brightness range 1, such that the microbe 1 characterized with the standard brightness range 1 and the microbe 2 characterized with the standard brightness range 2 are classified.

Alternatively, as illustrated in the histogram 2 (202), the standard brightness range 1 including both the peak 1 derived from the image of the microbe 1 and the peak 2 derived from the image of the microbe 2 is determined, and the standard brightness range 2 including the peak 2 without including the peak 1 is set, thereby making it possible to be classified into all microbes contained in the image, the microbe 2 characterized with the standard brightness range 2, and the microbe 1 obtained by subtracting the microbe 2 from all microbes.

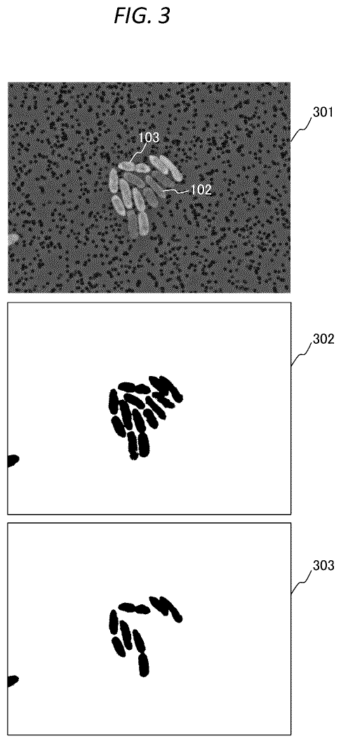

FIG. 3 illustrates an example in which the microbial image is classified based on a difference in brightness of the electron microscope image by using image analysis software, and an image mask is generated for each classification. As illustrated in the schematic diagram (101) of FIG. 1, in an electron microscope image (301) of a microbial specimen, the microbe 1 (102) having weak staining and low image brightness and the microbe 2 (103) having strong staining and high image brightness are mixed.

As illustrated in the histogram 2 (202) of FIG. 2, a mask (302) is a mask image generated in such a manner that the brightness range of the equipment is excluded, the image is binarized by setting a threshold value from the standard brightness range 1 including both the peak 1 derived from the image of the microbe 1 and the peak 2 derived from the image of the microbe 2, and then particles significantly smaller than the microbes 1 and 2 are removed by using a particle analysis algorithm. As illustrated in the histogram 2 (202) of FIG. 2, a mask (303) is a mask image generated in such a manner that the image is binarized by setting a threshold value from the standard brightness range 2 including the peak 2 without including the peak 1, and then particles significantly smaller than the microbe are removed by using a particle analysis algorithm. By using the image analysis software, the individual microbe classified into the mask 1 and the mask 2 can be identified, and the number of microbes, a microbial image area, and the number of pixels can be obtained to calculate a ratio.

As described above, the microbial specimen is stained to generate the backscattered electron image, the brightness profile with respect to the image brightness of the microbe 1 (104) having weak staining and low image brightness and the microbe 2 (105) having strong staining and high image brightness is acquired, and the microbial image is classified by the image brightness range, such that the ratio of the microbe 1 and the microbe 2 in the specimen can be easily calculated.

In the first embodiment, while the difference in the staining intensity of the microbe is set to two types, and two types of standard brightness ranges in the brightness profile with respect to the image brightness are determined, the types of standard brightness range may be two or more.

### SECOND EMBODIMENT

### Viability Test of Microbe

A second embodiment describes an example in which the ratio of the microbe obtained by the method of the present invention is compared with the viability of the microbe obtained by the colony forming unit, which is a method of related art of a viability test, and a flow cytometry method which is a non-culture method.

As an example, bacteria (Akkermansia muciniphila) are cultured anaerobically on an agar medium for 48 hours and isolated. After that, the isolated bacteria are suspended at bacterial concentration of ˜10¹⁰ CFU/mL in a storage medium containing an antioxidant, and is frozen and freeze-dried at −80° C. for 24 hours.

After being frozen and freeze-dried, the bacteria are stained with PTA of 10% for 5 minutes at 37° C., the bacterial suspension is smeared on a slide glass by centrifugation and dried at a room temperature, and SEM observation is performed at an acceleration voltage of 10 kV. Images of 500 bacterial cells are obtained, classified into two types according to the staining intensity, and the ratio is obtained.

In the colony forming unit, the bacteria after freezing and freeze-drying are diluted in 10 stages under anaerobic conditions by using anaerobic PBS, seeded on a Colombian blood agar plate, and cultured at 37° C. for 48 to 72 hours. The viability of bacteria is calculated as a ratio of the number of bacteria colonized by the culture to the number of bacteria at the start of the culture.

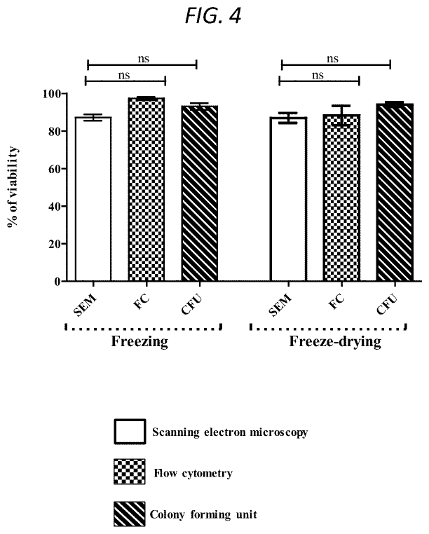

FIG. 4 illustrates a result obtained by measuring the viability of the bacteria after freezing and freeze-drying with the method of the present invention (Scanning electron microscopy), the flow cytometry method, or the colony forming unit. As a result of comparison, there is no statistically significant difference in the viability obtained by the three types of methods, either after freezing or after freeze-drying.

As another example, bacteria (Akkermansia muciniphila) are suspended in a Mueller Hinton medium (MHB) or a medium containing an antioxidant, and then left at a room temperature for one hour under anaerobic or aerobic conditions.

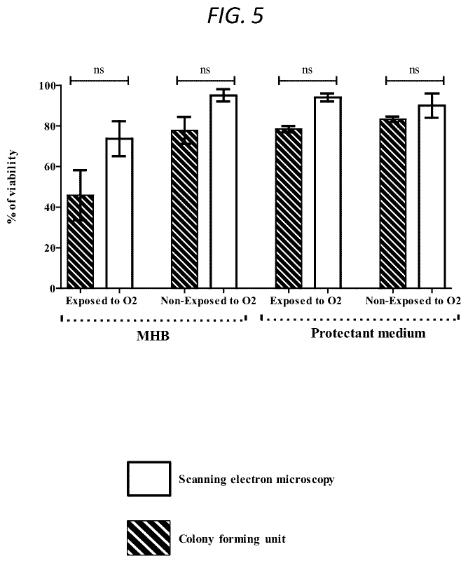

FIG. 5 illustrates results of a ratio of bacteria obtained by the method of the present invention and viability obtained by the colony forming unit, in the same manner as that of the above-described example. As a result of comparison, there is no statistically significant difference in the viability obtained by the two methods under either condition.

As illustrated in these examples, a correlation is found between the viability obtained by the viability test method of related art for the microbe and the ratio of the microbe obtained by the method of the present invention. In the method of the present invention, by observing the morphology of the individual microbe with the resolution of the electron microscope, it is also possible to analyze presence or absence of the antioxidant in the medium and the abnormal morphology caused by oxygen exposure. That is, it can be seen that according to the embodiment, the type and morphological characteristic of the microbe can be observed with the electron microscope and the viability can be evaluated.

### THIRD EMBODIMENT

A third embodiment describes an example of quantifying an effect of performing treatment having the bactericidal action on a microbe by analyzing an electron microscope image of the microbe stained with PTA.

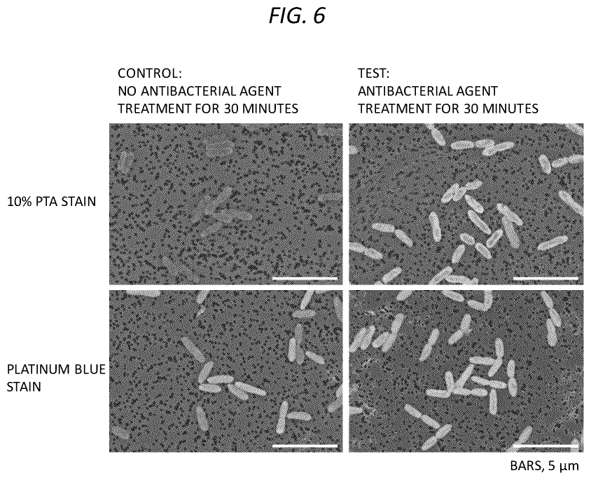

As an example, in FIG. 6, a Pseudomonas aeruginosa strain is used as a material, bacteria in a control group not treated with the antibacterial agent and bacteria in a test group treated with antibacterial agent colistin are cultured at 37° C. for 30 minutes, the bacteria are collected on a track-etched membrane and fixed with glutaraldehyde of 2.5%, a specimen is prepared by performing electronic staining for 2 minutes by using a PTA aqueous solution having weight concentration of 10% as a stain and a platinum blue aqueous solution as a comparison, and a backscattered electron image is obtained by SEM observation at an acceleration voltage of 5 kV.

In the specimen stained with the PTA aqueous solution, an image of bacteria having low image brightness is observed in the control group, and an image of bacteria having high image brightness is observed in the test group. On the other hand, in the specimen stained with the platinum blue aqueous solution, the image brightness of the bacteria in both the control group and the test group is high, and no significant difference is observed between the control group and the test group.

As another stain not shown in the drawing, in a specimen stained with a PTA aqueous solution whose pH is adjusted to neutral, the brightness of the bacterial image in the test group is as low as in the control group. On the other hand, in an aqueous solution of sodium tungstate or an aqueous solution of ammonium molybdate, no positive stained image is observed in both the control group and the test group. Accordingly, it can be seen that no significant difference is observed between the control group and the test group in these three types of stains.

As described above, by selecting the PTA aqueous solution having the weight concentration of 10% as a stain, the bacteria in the control group not treated with the antibacterial agent and the bacteria in the test group treated with the antibacterial agent and affected thereby can be stained based on the difference in image brightness.

In FIG. 6, an influence of colistin with respect to Pseudomonas aeruginosa is detected by the PTA staining, but a target of the embodiment is not limited to this combination. As another example, when Escherichia coli or Pseudomonas aeruginosa is treated with β-lactam antibiotic imipenem having the bactericidal action, the bacteria having high image brightness is increased by the PTA staining.

As described above, when an electronic stain that increases a stain intensity of a microbe sensitive to the bactericidal action is selected, it is possible to classify images of bacteria receiving damage by the bactericidal action and bacteria not receiving the damage based on a difference in electron microscope image brightness. Particularly, strong PTA staining in colistin-treated bacteria is considered to be related to the life and death caused by the damage in the membrane and the cell wall. That is, by classifying and quantifying the bacteria stained with PTA, a bactericidal effect of the antibacterial agent colistin can be analyzed and evaluated.

The embodiment is not limited to this example, and when a user takes advantage of the fact that the intensity of the electron staining changes depending on a state of the microbe at the time of performing treatment that affects the microbe, an image of the control group and an image of the test group are obtained, a microbial image brightness range that characterizes a microbial image of the control group and a microbial image brightness range that characterizes a microbial image of the test group having brightness different from that of the control specimen are determined, and the microbial images of the control group and the test group are classified and the ratio is obtained, such that the effect of the treatment affecting the microbe can be analyzed and evaluated.

For example, as the action of the antibacterial agent, many antibacterial agents that affect a cell wall of bacteria, inhibit growth of the bacteria, or show the bactericidal action are developed. Since a human cell does not have a cell wall, a compound that selectively acts on the cell wall of bacteria is expected to have a low side effect on humans. The method of this embodiment can be used to evaluate the effect of such compounds, or to screen a new compound based on an action mechanism.

In order to treat an infectious disease and control infection in hospital, it is important to quickly determine whether causative bacteria are resistant or not, and a drug sensitivity test is performed for each bacterium isolated from a patient. In a method of related art, resistance is determined from a value of minimum inhibitory concentration (MIC) that inhibits the growth of bacteria, and it takes about one day for culturing to obtain the MIC. On the other hand, in this embodiment, it is possible to analyze presence or absence of damage caused by an influence of the antibacterial agent, and to quickly determine the sensitivity of the causative bacteria.

As another example, this embodiment can also be used to evaluate a bacteriophage that infects bacteria and causes bacteriolytic action. A phage destroys a membrane and a peptidoglycan to kill bacteria. Phage therapy is being reviewed as one of the treatments for multidrug-resistant bacteria, and requires a method for rapidly selecting a combination of phages specific to the causative bacteria from a library of phages in the environment or artificially modified phages. In this embodiment, a bacteriolytic property of the phage is evaluated, thereby being able to be used for phage screening.

As another example, when the microbe is exposed to heat or oxygen by including not only bacteria but also fungi and archaea, and deterioration in the viability caused by microbial damage is evaluated, the evaluation can be used to select storage conditions of the microbe or a sterilization method.

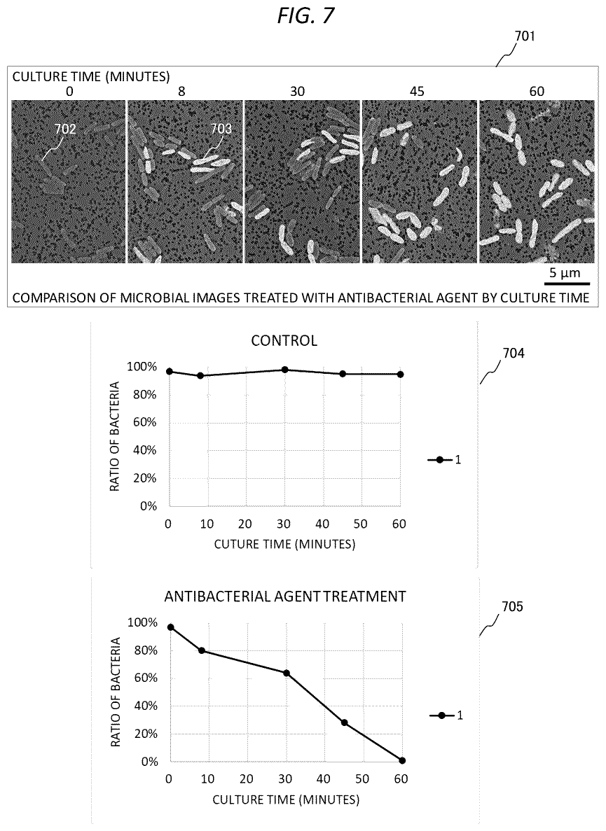

As a specific example of analysis of the treatment that affects the microbe, FIG. 7 shows a result of treating Pseudomonas aeruginosa with the antibacterial agent colistin and analyzing a change over time of the influence of colistin by the bacterial image brightness.

A Pseudomonas aeruginosa strain whose colistin sensitivity is known from an MIC value measured by a method of related art and a Pseudomonas aeruginosa strain whose colistin resistance is known are used. A bacterial solution suspended in a medium at initial concentration of 10⁶/ml is treated with colistin of 2 mg/L, which is concentration that distinguishes between sensitive bacteria and resistant bacteria by the method of related art, is treated and cultured at 37° C., and a certain amount of the bacterial solution is sampled over time.

A polycarbonate track-etched membrane having a hole diameter of 0.2 μm is used as specimen equipment for the SEM observation of bacteria, and bacteria are uniformly collected in a certain area on the membrane. Conductivity is given to the specimen by pre-vaporizing platinum-palladium on a surface of the membrane.

After washing a medium component on a bacterial collection surface with physiological saline, bacteria are treated and fixed for 5 minutes with a glutaraldehyde fixing solution of 2.5% having a protein cross-linking effect in order to prevent the morphological change of the bacteria. After washing the excess fixing solution with water, the bacteria are treated with the PTA aqueous solution having the weight concentration of 10% for 2 minutes and stained. After washing the excess stain with water, the bacteria are dried on the track-etched membrane to form a specimen for the SEM observation.

SEM observation conditions are set to an acceleration voltage of 5 kV, backscattered electron detection, and magnification of 7,000 times. The reason why the acceleration voltage is set to 5 kV is that a hole image of the track-etched membrane by an electron beam transmitted through a bacterial cell does not overlap with an image of Pseudomonas aeruginosa. The reason for setting the magnification to 7000 times is to observe the morphology of Pseudomonas aeruginosa, and the embodiment is not limited thereto.

As an observation step, before observing the bacterial specimen, a brightness standard sample is used and brightness contrast adjustment of the SEM is adjusted to be constant so that the brightness contrast adjustment of the SEM is made constant in the image acquisition of all the specimens. Accordingly, image analysis to be performed later becomes easier.

A series of backscattered electron images (701) in FIG. 7 is an example of an image acquired by SEM observation using a colistin-sensitive Pseudomonas aeruginosa strain. A backscattered electron image (702) is an example of an image obtained by SEM observation of the microbe 1 observed in a specimen at 0 minute of culture. The backscattered electron image (702) is colistin-unaffected Pseudomonas aeruginosa, and is the same as an untreated control specimen. A backscattered electron image (703) is an example of an image obtained by SEM observation of the microbe 2 observed after 8 minutes of culture, and is Pseudomonas aeruginosa whose membrane and cell wall are damaged by the influence of colistin and stained with PTA.

A brightness profile with respect to the brightness of the image (702) of the microbe 1 is acquired to determine the image brightness range 1 (108) illustrated in FIG. 1. The brightness profile regarding the brightness of the image (703) of the microbe 2 is acquired to determine the image brightness range 2 (107) illustrated in FIG. 1.

Next, SEM observation is performed on each specimen from which the series of backscattered electron image (701) is obtained and a specimen in the control group prepared at the same timing, and twenty-five images per specimen and images of 300 to 500 bacterial cells are acquired. The number of images taken and the number of bacterial cells to be imaged are not limited to the above-described number as long as reproducibility of quantitative analysis can be ensured.

A microbial image region is extracted from all the images taken of each specimen, and is classified into the microbe 1 characterized with the image brightness range 1 (108) and the microbe 2 characterized with the image brightness range 2 (107), thereby identifying whether an individual microbe is an individual not affected by colistin or an individual whose membrane and cell wall are damaged by the influence of colistin. The classified individuals are counted, and a ratio of the microbe 1 is calculated by counting the total number of microbes 1 and 2 as the total number of microbes. Since the microbe 1 is not affected by the influence of colistin and is considered to be alive, a change in the ratio of the microbe 1 corresponds to a change in viability.

A graph (704) shows a change over time in the ratio of the microbe 1 contained in the bacteria sampled from the control group, and a graph (705) shows a change over time in the ratio of the microbe 1 contained in the bacteria sampled from the test group treated with colistin. In the control group, the ratio of the microbe 1 hardly changes, but in the test group, the ratio of the microbe 1 is reduced to 80% after 8 minutes, 60% after 30 minutes, and 0% after 60 minutes. This result indicates that the Pseudomonas aeruginosa strain to be analyzed is sensitive to colistin.

On the other hand, when colistin-resistant Pseudomonas aeruginosa strain is treated with colistin in the same manner as the sensitive bacteria, the ratio of the microbe 1 in the control group corresponding to the graph (704) and the ratio of the microbe 1 in the test group corresponding to the graph (705) are hardly reduced, and are about 95% even after 60 minutes. This result indicates that colistin has no influence on the Pseudomonas aeruginosa strain to be analyzed.

As described above, it is possible to show a difference between the sensitive bacteria and the resistant bacteria by quantitatively analyzing and comparing the influence of colistin treatment on Pseudomonas aeruginosa, and the result of the sensitivity and resistance determination is consistent with the determination based on the MIC value which is the method of related art.

While it takes about one day for culturing to obtain the MIC with the method of related art, according to the embodiment, it is possible to quickly determine the sensitivity and resistance of the causative bacteria by analyzing the presence or absence of the morphological damage caused by the influence of the antibacterial agent.

Here, characteristics of the specimen in the embodiment will be described. Other viability tests using a microscopic observation technique are disclosed, and a life-and-death determination reagent dedicated for the optical microscope is based on the enzyme activity of the living cell and selective permeability of the cell membrane, and living at the time of observation is a prerequisite for staining. On the other hand, since a target to be stained with PTA is morphologically damaged in the process leading to killing bacteria, the target can be stained and observed even after being fixed with glutaraldehyde and the like and stored, thereby improving convenience of the viability test. Since a desktop SEM can be used in recent years, the desktop SEM can be applied to daily microbial tests such as an infectious disease test for a microbe, environmental monitoring, and a food safety test.

1-15. (canceled)

16. A microbial image analysis method, comprising:
a first step for obtaining an image of a specimen in which a sample including microbes is stained, by using an electron microscope;
a second step for obtaining a brightness profile regarding a brightness distribution range of the image;
a third step for setting a first standard brightness range which meets a first condition regarding a brightness out of the profile up as a region where a first microbe group exists, and setting a second standard brightness range which meets a second condition regarding a brightness out of the profile up as a region where a second microbe group exists; and
a fourth step for identifying individual microbes which exist in each of the first and the second standard brightness ranges and/or for calculating a ratio of the microbe which exists in each of the first and the second standard brightness ranges, wherein
the first condition includes a first peak of the brightness profile and does not include a second peak which is different from the first peak,
the second condition does not include the first peak and includes the second peak, and
he fourth step is for identifying the life or death of individual microbe and/or individual microbes which are morphologically damaged in each of the first and the second standard brightness ranges and/or for calculating microbial viability and/or a ratio of microbe which is morphologically damaged based on the number of microbes and/or a microbial image area, and the number of image pixels in each of the first and the second standard brightness ranges.

17. The microbial image analysis method according to claim 16, wherein
the specimen is a smear specimen which is prepared from a microbial suspension in order to prevent microbes and solids from overlapping each other.

18. The microbial image analysis method according to claim 16, wherein
the first step is for performing a chemical fixation process on microbes using at least one of glutaraldehyde, formalin, and alcohol in order to stop a morphological change and preserve a microbial shape.

19. The microbial image analysis method according to claim 16, wherein
the first step is for staining the sample by using a phosphotungstic acid solution.

20. The microbial image analysis method according to claim 19, wherein
the phosphotungstic acid solution is an acidic solution which is adjusted from pH 0.0 to pH 7.0.

21. The microbial image analysis method according to claim 19, wherein
the phosphotungstic acid solution is an acidic solution which is adjusted from pH 0.0 to pH 3.0.

22. The microbial image analysis method according to claim 19, wherein
the concentration of the phosphotungstic acid solution is from 0.1% to 20%.

23. The microbial image analysis method according to claim 19, wherein
the concentration of the phosphotungstic acid solution is from 2% to 10%.

24. A kit for implementing the microbial image analysis method according to claim 19, comprising:
a solution containing chemical fixasives which contains at least one of glutaraldehyde, formalin, and alcohol;
a stain which contains at least the phosphotungstic acid; and
a washing solvent which contains at least one of a physiological saline, water, and a buffer solution.

25. A microbial image analysis method, comprising:
a first step for obtaining a first image of a control specimen in which a sample including microbes is stained without executing a treatment which affects microbes, by using an electron microscope;
a second step for obtaining a second image of a test specimen in which a sample including microbes is stained with executing a treatment which affects microbes, by using the electron microscope;
a third step for obtaining a brightness profile regarding a brightness of the first image, and a first peak of the brightness of the profile; and
a fourth step for obtaining a brightness profile regarding a brightness of the second image, and a second peak of the brightness of the profile.

26. The microbial image analysis method according to claim 25, wherein
the third step is for obtaining a first standard brightness range including the first peak based on microbial species, a type of treatment which affects microbes and a type of stain, and
the fourth step is for obtaining a second standard brightness range including the second peak based on microbial species, a type of treatment which affects microbes and a type of stain.

27. The microbial image analysis method according to claim 26, further comprising:
a fifth step for obtaining the number of microbes, a microbial image area, and/or the number of pixels and calculating a ratio of microbe which exists in each of the first and the second standard brightness range, and quantifying an effect of treatments that affect microbes by comparing the control specimen with the test specimen.

28. The microbial image analysis method according to claim 25, wherein
the treatment which affects the microbes is to give the microbes at least one of an antimicrobial, heat, oxygen, a detergent, and a bacteriophage.

29. The microbial image analysis method according to claim 26, wherein
the stain is phosphotungstic acid.
